SEQUENCE LISTING (20 sequences)

SEQ ID NO: 1 - DNA, 68 bp, Artificial Sequence
  Synthetic Nucleotide
aggctggattttttatttacctttttttaggtaaaacgacggccagcctttttttttttttccgtcgt

SEQ ID NO: 2 - DNA, 28 bp, Artificial Sequence
  Synthetic Nucleotide
aggctggaggtaaaacgacggccagcct

SEQ ID NO: 3 - DNA, 55 bp, Artificial Sequence
  Synthetic Nucleotide
ggcgaggtaaaacgacggccagcctcgccgtttttttttttttttttgccgtcgt

SEQ ID NO: 4 - DNA, 15 bp, Artificial Sequence
  Synthetic Nucleotide
ctggccgtcgtttta

SEQ ID NO: 5 - DNA, 15 bp, Artificial Sequence
  Synthetic Nucleotide
ctggccgtagtttta

SEQ ID NO: 6 - DNA, 17 bp, Artificial Sequence
  Synthetic Nucleotide
gctggccgtcgttttac

SEQ ID NO: 7 - DNA, 17 bp, Artificial Sequence
  Synthetic Nucleotide
gctggccctcgttttac

SEQ ID NO: 8 - DNA, 17 bp, Artificial Sequence
  Synthetic Nucleotide
gctggcccccgttttac

SEQ ID NO: 9 - DNA, 19 bp, Artificial Sequence
  Synthetic Nucleotide
ggctggccgtcgttttacc

SEQ ID NO: 10 - DNA, 19 bp, Artificial Sequence
  Synthetic Nucleotide
ggctggccctcgttttacc

SEQ ID NO: 11 - DNA, 69 bp, Artificial Sequence
  Synthetic Nucleotide
aggctggattttttatttacctttttttaggtaaaacgacggccagcctttttttttttttccgtcgtt

SEQ ID NO: 12 - DNA, 17 bp, Artificial Sequence
  Synthetic Nucleotide
gctgggcgtcgttttac

SEQ ID NO: 13 - DNA, 17 bp, Artificial Sequence
  Synthetic Nucleotide
gctgggggtcgttttac

SEQ ID NO: 14 - DNA, 17 bp, Artificial Sequence
  Synthetic Nucleotide
gctggccgacgttttac

SEQ ID NO: 15 - DNA, 17 bp, Artificial Sequence
  Synthetic Nucleotide
gctggccgccgttttac

SEQ ID NO: 16 - DNA, 17 bp, Artificial Sequence
  Synthetic Nucleotide
gctggccggcgttttac

SEQ ID NO: 17 - DNA, 17 bp, Artificial Sequence
  Synthetic Nucleotide
gctggccgtggttttac

SEQ ID NO: 18 - DNA, 17 bp, Artificial Sequence
  Synthetic Nucleotide
gctggccgtagttttac

SEQ ID NO: 19 - DNA, 17 bp, Artificial Sequence
  Synthetic Nucleotide
gctggccgtccttttac

SEQ ID NO: 20 - DNA, 17 bp, Artificial Sequence
  Synthetic Nucleotide
gctggccgtcgctttac

(19) United States
(12) Patent Application Publication
(10) Pub. No.: US 2011/0256637 A1
(43) Pub. Date: Oct. 20, 2011

(54) Target Detection Using a Single-Stranded, Self-Complementary, Triple-Stem DNA Probe

(76) Inventors: Hyongsok Soh, Santa Barbara, CA (US); Yi Xiao, Goleta, CA (US)

(21) Appl. No.: 13/123,577

(22) PCT Filed: Oct. 13, 2009
(86) PCT No.: PCT/US09/60543

§ 371 Date: Jul. 1, 2011

Related U.S. Application Data
(60) Provisional application No. 61/107,991, filed on Oct. 23, 2008.

(51) Int. Cl. G01N 33/53; C07H 21/00; C40B 40/06

(52) U.S. Cl. 436/501; 506/16; 536/24.3

(57) ABSTRACT

Provided are novel single-stranded oligonucleotide probes that have a triple-stem configuration in the absence of target binding to the target binding sequence. The probes also have a fluorophore and a quencher. In the absence of target binding to the target binding sequence, these single-stranded oligonucleotide probes are capable of forming self-complementary duplexes such that the probe is in the triple-stem configuration and the fluorophore is positioned adjacent the quencher. In the presence of target binding to the target binding sequence, formation of the self-complementary duplexes is inhibited such that the probe is configured to position the fluorophore away from the quencher such that a signal of the fluorophore is detectable. Also provided are methods of using the probes.

### CROSS REFERENCE TO RELATED APPLICATION

This application claims priority to U.S. Provisional Patent Application No. 61/107,991 filed Oct. 23, 2008, which is incorporated herein by reference in its entirety and for all purposes.

### REFERENCE TO GOVERNMENT SUPPORT

This invention was made in part with government support under grants from the National Institutes of Health (Grant No. R21 EB008215), the Office of Naval Research (Grant No. N00014-08-1-0469), and the Institute for Collaborative Biotechnologies (Grant No. DAAD19-03-D-0004) from the U.S. Army Research Office. The government has certain rights in this invention.

### INTRODUCTION

Sequence-specific analysis of nucleic acids is important for genomic studies, disease diagnosis, and in vitro monitoring of biological processes. In particular, the detection of single nucleotide polymorphisms (SNPs) can serve as an important indicator of genetic predisposition towards specific disease states or drug responses, and there is a need for technologies suitable for rapid, sensitive, specific, and inexpensive SNP detection that is scaleable. Ideally, such method should be a single-step, single-component, reagentless, room temperature assay that is compatible with microarray technology for massive parallel analysis. Unfortunately, current technologies for SNP detection can only partially satisfy these requirements.

Standard enzymatic methods such as endonuclease digestion, primer extension, and ligation assays are complex, multi-step techniques that often require separation of the resultant products in order to determine the presence of the target sequence. These cumbersome requirements have hindered the scalability of these technologies to date, and have motivated the pursuit of simpler, fluorescence-based SNP detection assay including methods utilizing molecular beacon and binary probes.

Molecular beacons (MBs) are self-complementary, hairpin-shaped oligonucleotides containing fluorophore/quencher pairs which are suitable for rapid and scalable hybridization analysis. When a complementary target is introduced, probe hybridization disrupts the hairpin structure, segregating the fluorophore/quencher pair and thereby inducing an increase in fluorescence. MBs enable rapid, reagentless and quantitative SNP analysis in homogeneous solutions without the need for separation steps; however, this method's reliance on probe-target duplex melting temperature as the basis for discrimination between matched and mismatched targets limits the range of products that can be analyzed to those whose distinct melting temperatures can be distinguished via precise temperature control.

Binary probes make use of two different DNA probes that form relatively short (e.g. 7 to 10 nucleotide) duplexes when hybridized to adjacent sites of a target sequence. These short hybrids are sensitive to single nucleotide substitutions and generate a signal only in the presence of perfectly-matched targets; signal detection can be achieved via ligation reaction, fluorescence or colorimetric readouts, or resonance energy transfer. Binary probes produce specific, sensitive and reliable results without the need for precise temperature control; however, the method requires the addition of exogenous reagents.

### SUMMARY

Provided are novel single-stranded oligonucleotide probes that have a triple-stem configuration in the absence of target binding to the target binding sequence. The probes also have a fluorophore and a quencher. In the absence of target binding to the target binding sequence, these single-stranded oligonucleotide probes are capable of forming self-complementary duplexes such that the probe is in the triple-stem configuration and the fluorophore is positioned adjacent the quencher. In the presence of target binding to the target binding sequence, formation of the self-complementary duplexes is inhibited such that the probe is configured to position the fluorophore away from the quencher such that a signal of the fluorophore is detectable. Also provided are methods of using the probes.

Accordingly, in one embodiment, the single-stranded oligonucleotide probe includes a target binding sequence, a first hybridization sequence, a second hybridization sequence, a third hybridization sequence, a fourth hybridization sequence, a fluorophore, and a quencher. In these embodiments, in the absence of target binding to the target binding sequence, the first hybridization sequence and the second hybridization sequence form a first duplex and the third hybridization sequence and the fourth hybridization sequence form a second duplex such that the probe is in a triple-stem configuration and the fluorophore is positioned adjacent the quencher. In this configuration, the emission of the fluorophore is suppressed by the quencher. In some cases, the first duplex and the second duplex are adjacent each other when the probe is in the triple-stem configuration. Alternatively, in the presence of target binding to the target binding sequence, formation of duplexes between the hybridization sequences is inhibited by specific interaction of the target with the target binding sequence such that the probe is configured to position the fluorophore away from the quencher. In this configuration, the emission of the fluorophore is not suppressed by the quencher and the fluorophore emits a detectable signal.

In certain embodiments, the target binding sequence comprises at least a portion of the first hybridization sequence. In other embodiments, the target binding sequence comprises at least a portion of the second hybridization sequence. In still other embodiments, the target binding sequence comprises at least a portion of the second hybridization sequence and at least a portion of the third hybridization sequence.

In some cases, the probes further comprise a fifth hybridization sequence and a sixth hybridization sequence. In these cases, in the absence of target binding to the target binding sequence, the fifth hybridization sequence and the sixth hybridization sequence may form a third duplex. In particular embodiments, the first duplex is flanked by the second duplex and the third duplex. In some cases, the second duplex and the third duplex are separated by a hairpin structure. In certain embodiments, the first duplex, the second duplex and the third duplex together comprise about 10 to about 30 base pairs, such as about 21 base pairs, including embodiments where the first duplex, the second duplex and the third duplex together comprises 21 base pairs.

In certain embodiments that further comprise a fifth hybridization sequence and a sixth hybridization sequence, the target binding sequence may comprise at least a portion of the second hybridization sequence, at least a portion of the third hybridization sequence, and at least a portion of the sixth hybridization sequence.

In certain embodiments, the specificity of the target binding sequence for target is such that the target binding sequence only hybridizes target when perfectly complementary to the target. In particular cases, the target binding sequence has a discrimination factor of about 5 or more, where the discrimination factor is the ratio of the net fluorescence intensity obtained with the perfectly-matched target to that obtained with a mismatched target, after subtraction of background fluorescence.

In some cases, the target binding sequence comprises about 10 to about 30 contiguous nucleotides complementary to the target, such as about 15 to about 19 contiguous nucleotides complementary to the target, including embodiments where the target binding sequence comprises 17 contiguous nucleotides complementary to the target.

In particular embodiments, the quencher is attached to the probe at a position within the target nucleotide sequence, and wherein the fluorophore is attached to the probe at an end of the probe sequence. In alternative embodiments, the fluorophore is attached to the probe at a position within the target nucleotide sequence, and wherein the quencher is attached to the probe at an end of the probe sequence. In some cases, the probe is immobilized on a surface of a substrate. In addition, the substrate may comprise an addressable array of a plurality of the probes.

In another embodiment, a method for detecting a target in a sample is provided. The method includes: (a) contacting a single-stranded triple-stem probe, as described herein, with the sample under hybridization conditions, whereby the target selectively hybridizes to the target binding sequence to form a target-probe hybrid; and (b) detecting the presence or absence of the target-probe hybrid, wherein the detecting comprises detecting fluorescent emission from the fluorophore.

In certain embodiments, the method may be used to detect concentration ranges of target in the sample from about 1 nM and about 300 nM, such as from about 2 nM and about 150 nM, including from about 3 nM to about 100 nM.

Further provided are methods for detecting the presence of a single nucleotide polymorphism in a target. In these embodiments, the method includes contacting a single-stranded triple-stem probe, as described herein, with a sample comprising the target under hybridization conditions. In this method, the target binding sequence includes a single nucleotide mismatch, and the target selectively hybridizes to the target binding sequence to form a target-probe hybrid. The method further includes detecting the presence or absence of the target-probe hybrid, where the presence of the target-probe hybrid indicates the presence of a single nucleotide polymorphism in the target. In these cases, the single nucleotide polymorphism in the target is complementary to the single nucleotide mismatch in the target binding sequence.

### BRIEF DESCRIPTION OF THE FIGURES

The disclosure is best understood from the following detailed description when read in conjunction with the accompanying drawings. It is emphasized that, according to common practice, the various features of the drawings are not necessarily to-scale. In some cases, the dimensions of the various features may have been arbitrarily expanded or reduced for clarity. Included in the drawings are the following figures.

FIG. 1 shows a schematic drawing of the mechanism of the triple-stem probe.

FIG. 2 shows emission spectra of the triple-stem probe (1) (0.5 μM) following incubation at room temperature with a perfectly-matched (PM) target (2), single-base mismatched (1 MM) target (3), two-base mismatched (2 MM) target (4), or in the absence of target.

FIG. 3, left, shows thermal denaturation curves of the triple-stem probe (1) (0.5 μM) only, or hybridized with a perfectly-matched (PM) target (2), a single-base mismatched (1 MM) target (3), or a two-base-mismatched (2 MM) target. FIG. 3, right, shows a graph of the kinetics of the triple-stem probe (1) (0.5 μM) only, or hybridized with perfectly-matched (PM), single-base (1 MM) or two-base-mismatched (2 MM) targets, monitored at room temperature.

FIG. 4, left, shows emission spectra of the triple-stem probe (1) (0.5 μM) only, probe-single-base mismatched target (3) duplexes, or probe-perfectly-matched target (2) duplexes at different concentrations, recorded at room temperature. FIG. 4, right, shows a calibration curve of perfectly-matched target (2) and single-base mismatched target (3) for the triple-stem probe (1). The signal change demonstrates sensitive discrimination ability over wider target concentration range. The inset shows the dependence of discrimination factor of 17-base targets in the presence of 0.5 μM of the triple-stem probe.

### DEFINITIONS

Unless defined otherwise, all technical and scientific terms used herein have the meaning commonly understood by a person skilled in the art to which this invention belongs. As used herein, the following terms have the following meanings unless otherwise indicated.

As used herein, different oligonucleotide probe structures, such as those that exist in the presence or absence of a target, may be as referred to as “conformations.”

“Target” refers to any molecule that specifically binds to a probe of the present disclosure. These include carbohydrates, nucleic acids, peptides, proteins, lipids, small molecules, inorganic or organic ions.

The term “probe” as used herein refers to a biopolymer that specifically binds to a target of the present disclosure. Probes may include nucleic acids (RNA or DNA), aptamers, etc.

The particular use of terms “nucleic acid,” “oligonucleotide,” and “polynucleotide” should in no way be considered limiting and may be used interchangeably herein. “Oligonucleotide” is used when the relevant nucleic acid molecules typically comprise less than about 100 bases. “Polynucleotide” is used when the relevant nucleic acid molecules typically comprise more than about 100 bases. Both terms are used to denote DNA, RNA, modified or synthetic DNA or RNA (including but not limited to nucleic acids comprising synthetic and naturally-occurring base analogs, dideoxy or other sugars, thiols or other non-natural or natural polymer backbones), or other nucleobase containing polymers. Accordingly, the terms should not be construed to define or limit the length of the nucleic acids referred to and used herein.

Oligonucleotides of the present disclosure may be single-stranded, double-stranded, triple-stranded, or include a combination of these conformations. Generally oligonucleotides contain phosphodiester bonds, although in some cases, as outlined below, nucleic acid analogs are included that may have alternate backbones, comprising, for example, phosphoramide, phosphorothioate), phosphorodithioate, O-methylphophoroamidite linkages, and peptide nucleic acid backbones and linkages. Other analog nucleic acids include those with positive backbones, non-ionic backbones, and non-ribose backbones. Nucleic acids containing one or more carbocyclic sugars are also included within the definition of nucleic acids. These modifications of the ribose-phosphate backbone may be done to facilitate the addition of additional moieties such as labels, or to increase the stability and half-life of such molecules in physiological environments.

The term “nucleic acid sequence” or “oligonucleotide sequence” refers to a contiguous string of nucleotide bases and in particular contexts also refers to the particular placement of nucleotide bases in relation to each other as they appear in a oligonucleotide.

The terms “complementary” or “complementarity” refer to polynucleotides (i.e., a sequence of nucleotides) related by base-pairing rules. For example, the sequence “5′-AGT-3′,” is complementary to the sequence “5′-ACT-3′”. Complementarity may be “partial,” in which only some of the nucleic acids' bases are matched according to the base pairing rules, or there may be “complete” or “total” complementarity between the nucleic acids. The degree of complementarity between nucleic acid strands can have significant effects on the efficiency and strength of hybridization between nucleic acid strands under defined conditions. This is of particular importance for methods that depend upon binding between nucleic acids.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence.

Hybridization is carried out in conditions permitting specific hybridization. The length of the complementary sequences and GC content affects the thermal melting point Tₘ of the hybridization conditions necessary for obtaining specific hybridization of the target site to the target nucleic acid. Hybridization may be carried out under stringent conditions. The phrase “stringent hybridization conditions” refers to conditions under which a probe will hybridize to its target subsequence, typically in a complex mixture of nucleic acid, but to no other sequences at a detectable or significant level. Stringent conditions are sequence-dependent and will be different in different circumstances. Stringent conditions are those in which the salt concentration is less than about 1.0 M sodium ion, such as less than about 0.01 M, including from about 0.001 M to about 1.0 M sodium ion concentration (or other salts) at a pH between about 6 to about 8 and the temperature is in the range of about 20° C. to about 65° C. Stringent conditions may also be achieved with the addition of destabilizing agents, such as but not limited to formamide. For high stringency hybridization, the triple-stem oligonucleotide probes, as described herein, will specifically bind to a target with a discrimination factor of about 3 or more, such as about 5 or more, about 7 or more, about 10 or more, such as about 15 or more, including about 20 or more, for example about 25 or more.

The terms “thermal melting point”, “melting temperature” or “Tₘ” refer herein to the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium). In some cases, the term “T_{d}” is used to define the temperature at which at least half of the probe dissociates from a perfectly matched target nucleic acid.

The formation of a duplex molecule with all perfectly formed hydrogen-bonds between corresponding nucleotides is referred as “matched” or “perfectly matched”, and duplexes with single or several pairs of nucleotides that do not correspond are referred to as “mismatched.” Any combination of single-stranded RNA or DNA molecules can form duplex molecules (DNA:DNA, DNA:RNA, RNA:DNA, or RNA:RNA) under appropriate experimental conditions.

The phrase “selectively (or specifically) hybridizing” refers to the binding, duplexing, or hybridizing of a molecule only to a particular nucleotide sequence under stringent hybridization conditions when that sequence is present in a complex mixture (e.g. total cellular or library DNA or RNA).

Those of ordinary skill in the art will readily recognize that alternative hybridization and wash conditions can be utilized to provide conditions of similar stringency and will recognize that a combination of hybridization parameters can provide for a desired stringency.

The term “fluorophore” refers to any molecular entity that is capable of absorbing energy of a first wavelength and re-emit energy at a different second wavelength. Exemplary fluorophores include, but are not limited to CAL Fluor Red 610 (FR610; Biosearch Technologies, Novato, Calif.), fluorescein isothiocyanate, fluorescein, rhodamine and rhodamine derivatives, coumarin and coumarin derivatives, cyanine and cyanine derivatives, Alexa Fluors (Molecular Probes, Eugene, Oreg.), DyLight Fluors (Thermo Fisher Scientific, Waltham, Mass.), and the like.

The terms “quencher” or “dark quencher” refer to a substance that absorbs excitation energy from a fluorophore and dissipates that energy as heat. Dark quenchers are used in conjunction with fluorophores, such that when the quencher is positioned adjacent the fluorophore or at a distance sufficiently close to the fluorophore, the emission of the fluorophore is suppressed. However, when the quencher is positioned away from the fluorophore or at a distance sufficiently far from the fluorophore, the emission of the fluorophore is not suppressed, such that a signal of the fluorophore is detectable. Exemplary quenchers include, but are not limited to Black Hole Quencher (BHQ; Biosearch Technologies, Novato, Calif.), Dabsyl (dimethylaminoazosulphonic acid), Qxl quenchers (AnaSpec Inc., San Jose, Calif.), Iowa black FQ, Iowa black RQ, and the like.

The term “sample” as used herein relates to a material or mixture of materials, typically, although not necessarily, in fluid form, containing one or more components of interest.

The terms “optional” or “optionally” as used herein mean that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

Before the present invention is described in greater detail, it is to be understood that this invention is not limited to the particular embodiments described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, it will be readily apparent to one of ordinary skill in the art in light of the teachings herein that certain changes and modifications may be made thereto without departing from the spirit and scope of the appended claims. Any recited method can be carried out in the order of events recited or in any other order which is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. To the extent such publications may set out definitions of a term that conflicts with the explicit or implicit definition of the present disclosure, the definition of the present disclosure controls. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### DETAILED DESCRIPTION OF EXEMPLARY EMBODIMENTS

Provided are single-stranded oligonucleotide probes that have a triple-stem configuration in the absence of target binding to the target binding sequence. The probes also have a fluorophore and a quencher. In the absence of target binding to the target binding sequence, these single-stranded oligonucleotide probes are capable of forming self-complementary duplexes such that the probe is in the triple-stem configuration and the fluorophore is positioned adjacent the quencher. In the presence of target binding to the target binding sequence, formation of the self-complementary duplexes is inhibited such that the probe is configured to position the fluorophore away from the quencher such that a signal of the fluorophore is detectable. Also provided are methods of using the probes.

Below, the subject single-stranded, self-complementary, triple-stem oligonucleotide probes are described first in greater detail, followed by a review of the various methods in which that the probes may find use, as well as a discussion of various representative applications in which the subject probes and methods find use.

### Single-Stranded, Self-Complementary, Triple-Stem Oligonucleotide Probes

Provided are probes and detectors capable of specifically identifying nanomolar concentrations of biomolecules in solution. The probes can be made as single-stranded oligonucleotides constructed using techniques well-known to those of skill in the art, and contain internal sequences allowing the oligonucleotide strand to undergo intramolecular hybridization. This intramolecular hybridization results in the probe taking a secondary conformation termed a triple-stem. In certain embodiments, the detectors are constructed by linking an oligonucleotide probe to a surface of a substrate.

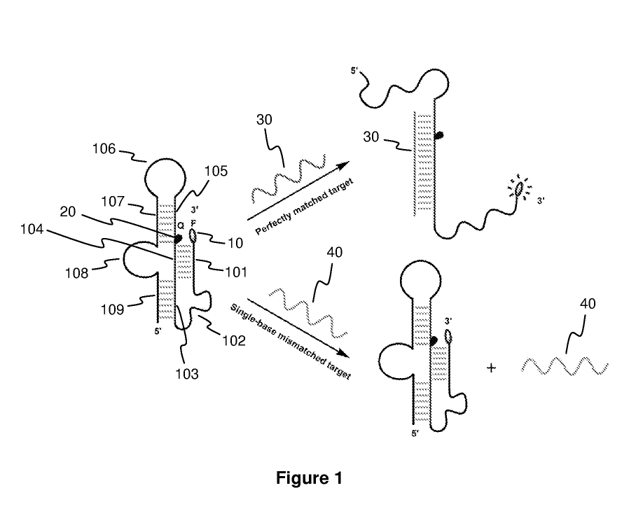

An exemplary triple-stem oligonucleotide probe is depicted in FIG. 1. An aspect of the triple-stem oligonucleotide probe of FIG. 1 is that the probe has a stem structure formed from three portions of the same single-stranded oligonucleotide. This triple-stem structure is formed through intramolecular hybridization between at least four internal hybridization sequences (IHSs). Internal hybridization between IHS101 and IHS104 forms a first duplex, and internal hybridization between IHS105 and IHS107 forms a second duplex. Optionally, the probe further comprises IHS103 and IHS109, which can hybridize to form a third duplex. IHS101 and IHS103 are separated by a loop structure formed by oligonucleotide sequence 102. IHS105 and IHS107 are separated by a loop structure formed by oligonucleotide sequence 106. In embodiments that further comprise IHS103 and IHS109, IHS107 and IHS109 are separated by a loop structure formed by oligonucleotide sequence 108. The first duplex and the second duplex may be adjacent each other. An aspect of the probes that further comprise IHS103 and IHS109 is that the first duplex may be flanked by the second duplex and the third duplex.

The triple-stem oligonucleotide probe further comprises a fluorophore 10 and a quencher 20. In some cases, the fluorophore 10 is coupled to one end of the oligonucleotide strand of the probe. In these cases, the quencher 20 is coupled to the oligonucleotide strand of the probe at an internal site, such that, in the absence of target binding to the target binding sequence, the internal hybridization between IHS101 and IHS104 positions the fluorophore 10 adjacent the quencher 20 such that the quencher 20 suppresses emission from the fluorophore 10. In alternative embodiments, the quencher 20 may be coupled to one end of the oligonucleotide strand of the probe, and the fluorophore 10 may be coupled to an internal site. Similarly, in these embodiments, in the absence of target binding to the target binding sequence, the internal hybridization between IHS101 and IHS104 positions the quencher 20 adjacent the fluorophore 10 such that the quencher 20 suppresses emission from the fluorophore 10.

In other embodiments, the triple-stem oligonucleotide probe is immobilized via an optional linker onto the surface of a substrate, e.g. through a chemical coupling or anchor. The optional linker may be any molecular moiety compatible as an adapter capable of coupling to both the substrate surface and the oligonucleotide strand forming the “triple-stem” structure of the probe, such as an oligonucleotide sequence, a peptide or amino acid, a sugar, etc. Suitable linkers are known to one of skill in the art. The probe, either directly or indirectly via the optional linker, is coupled to the substrate surface using techniques well-known to those of skill in the art. The end of the oligonucleotide strand of the probe that is not (in)directly coupled to the substrate surface is coupled to either the fluorophore 10 or the quencher 20, as described above.

The response to perfectly matched target 30 of the probe presented in FIG. 1 is a release of the restraint placed on the end of the probe coupled to the fluorophore 10 sufficient to allow fluorophore 10 to move a distance away from quencher 20, such that fluorophore 10 is no longer suppressed by quencher 20 and a detectable signal is observed. This is depicted schematically in FIG. 1 as a complete disruption of IHS hybridization in the probe allowing the fluorophore to be positioned away from the quencher resulting in a detectable emission from the fluorophore. These schematics should not however be construed as literally requiring complete disruption of base-pairing between complementary IHSs for fluorescence from the fluorophore to occur.

The probe can be designed so as to provide for discrimination between nucleic acid targets that differ by a single nucleotide in the target binding sequence. Thus, as shown in FIG. 1, binding of a perfectly matched target 30 to a probe is specific binding, allowing the probe to discriminate between a perfectly matched target 30 and other molecular entities that may be present in a sample, such as a single-base mismatched target 40.

Specific binding through complementary base-pairing between the probe sequence, or target binding sequence, and the perfectly matched target 30 results in a change in the structure of the probe allowing the fluorophore 10 to be positioned away from the quencher 20 such that a signal of the fluorophore 10 is detectable. Alternatively, if a sample contains a mismatched target, such as a single-base mismatched target 40, specific binding between the probe sequence, or target binding sequence, and the mismatched target 40 does not occur. In this case, the structure of the probe remains substantially the same and the fluorophore 10 remains in a position adjacent the quencher 20 such that the fluorescence of the fluorophore 10 is suppressed by the quencher 20.

The phrase binding “specifically” or “selectively,” refers to the interaction of a triple-stem oligonucleotide probe, as described herein, with a specific target in a manner that is determinative of the presence of the target in the presence or absence of a heterogeneous population of molecules that may include nucleic acids, proteins, and other biological molecules. Thus, under designated conditions, a specified triple-stem oligonucleotide probe binds to a particular target and does not bind in a significant manner to other molecules in the sample. Probes do not bind to a molecule in a detectable or significant manner when the interaction does not disrupt the intramolecular hybridization of the probe resulting in suppression of the fluorophore's emission by the quencher. In certain embodiments, the triple-stem oligonucleotide probes, as described herein, will specifically bind to a target with a discrimination factor of about 3 or more, such as about 5 or more, about 7 or more, about 10 or more, such as about 15 or more, including about 20 or more, for example about 25 or more.

Moreover, “specific binding” results in a disruption of intramolecular hybridization between probe nucleotide sequences resulting in a conformational change in the probe such that the fluorophore is positioned away from the quencher, such that a signal of the fluorophore is detectable. Thus, specific binding may be determined by titration of the probe with a target. Specific binding will allow an increase in signal with increasing amount of target contacted with the probe.

The following sections provide exemplary embodiments, including preferred embodiments, and additional disclosure allowing one of skill in the art to make and use the claimed invention. A detailed description of how to construct and use the systems of the disclosure is provided. Methods for using the systems are also discussed.

### I. Systems

Systems of the present disclosure include one or more species of triple-stem oligonucleotide probe described in more detail below. The probes are oligonucleotides that may be of any length, but are typically short oligonucleotides with ranges between 40 and 100 nucleotides, or between 50 and 75 nucleotides, such as between 60 and 70 nucleotides, for example 68 nucleotides. Oligonucleotide lengths of 50, 55, 60, 61, 62, 63, 64, 65, 66, 67, 68, 69 and 70, 75, 80, 85, 90, 95 and 100 or more residues are generally useful. The probes may recognize their targets by base-complementarity with the probe target binding sequence. While not an exhaustive list, in certain embodiments, the target may be an aptamer, antibody, receptor, or enzyme that specifically binds the probe.

Probes may be free in solution or, alternatively, may be attached by one end of the nucleotide chain to a surface of a substrate. In the absence of target, the fluorophore is held at distance in close proximity to the quencher, such as adjacent the quencher, by complementary base-pairing within the probe. Under conditions in the absence of target, the distance the fluorophore is held from the quencher is sufficient to minimize, suppress, or prevent the fluorophore from emitting a detectable signal. When target is present and binds to the probe, the internal hybridization pattern of the probe is disrupted. Disruption of the internal hybridization pattern allows the end of the nucleotide chain to which the fluorophore is attached to move to a distance further away from the quencher. Under conditions in the presence of target, the distance the fluorophore moves away from the quencher is sufficient to allow the fluorophore to emit a detectable signal.

Target may be removed and the probe regenerated using mild conditions that retain the integrity of the probe and allow the probe to re-establish the internal base pair hybridization pattern that suppresses the fluorescence of the fluorophore. In these embodiments, the probes are reusable, such that the probes may be regenerated as described above and reused any number of times, such as 2 or more times, including 3 or more times, for instance 5 or more times, or 10 times or more, while maintaining substantially the same ability to discriminate between perfectly matched targets and mismatched targets.

The sections that follow will provide instruction on the construction of triple-stem oligonucleotide probes, coupling the probes to the substrate surface, regeneration of the probes and the general use of the systems of the disclosure.

A. Triple-Stem Oligonucleotide Probes

Aspects of the presently disclosed systems include the triple-stem oligonucleotide probes, of which exemplary embodiments are schematically provided in FIG. 1. Triple-stem oligonucleotide probes are single-stranded nucleic acid molecules of variable length, depending upon the application and target molecule to be recognized. Using this disclosure, one of skill in the art may determine the length of probe to be used without undue experimentation, but as a guide ranges between 40 and 100 nucleotides, or between 50 and 75 nucleotides, such as between 60 and 70 nucleotides, for example 68 nucleotides are not uncommon with nucleotide lengths of 50, 55, 60, 61, 62, 63, 64, 65, 66, 67, 68, 69 and 70, 75, 80, 85, 90, 95 and 100 or more residues are generally useful.

Another characteristic of triple-stem oligonucleotide probes are internal hybridization sequences (IHSs). Each triple-stem oligonucleotide probe includes IHS sequences, where each IHS is complementary to another IHS of the probe and hybridizes to it in the absence of target binding to the probe. In certain embodiments, the triple-stem oligonucleotide probe includes one or more, such as two or more, including four or more, for example 6 or more IHS sequences. As will be discussed in more detail below, hybridization between IHSs positions the fluorophore in close proximity to the quencher such that the quencher suppresses the fluorescence of the fluorophore. When target binding to the probe occurs, IHS sequence hybridization is disrupted, allowing the fluorophore to be positioned at a distance away from the quencher such that the fluorophore emits a detectable signal. This alteration in the proximity of the fluorophore to the quencher in response to target binding to the probe provides systems of the disclosure their characteristic “signal on” response.

The triple-stem oligonucleotide probes also include at least one target binding sequence where specific binding of the target to the probe occurs. As will be discussed in greater detail below, the target binding sequence may be a continuous nucleotide sequence. For example, the target binding sequence may be one continuous nucleotide sequence that is complementary to a target nucleic acid molecule.

The characteristics briefly outlined above and additional characteristics of the probes of the present disclosure will be discussed in greater detail, below.

1. Probe Synthesis

As discussed herein, triple-stem oligonucleotide probes of the disclosure are single-stranded oligonucleotides that are typically between about 40 and 100 nucleotides, or between 50 and 75 nucleotides, such as between 60 and 70 nucleotides, for example 68 nucleotides are not uncommon with nucleotide lengths of 50, 55, 60, 61, 62, 63, 64, 65, 66, 67, 68, 69 and 70, 75, 80, 85, 90, 95 and 100 or more residues are generally useful. Both solution and solid phase techniques for synthesizing single-stranded oligonucleotides of this length are well known to those of skill in the art. In particular, methods of synthesizing oligonucleotides are found in, for example, Oligonucleotide Synthesis: A Practical Approach, Gait, ed., IRL Press, Oxford (1984), incorporated herein by reference in its entirety for all purposes.

Oligonucleotides may also be custom made and ordered from a variety of commercial sources known to persons of skill in the art. Purification of oligonucleotides, where necessary, may be performed for example by native acrylamide gel electrophoresis or by anion-exchange HPLC as described in Pearson and Regnier (1983) J. Chrom. 255:137-149.

a. Stem and Loop Structures

An aspect of the triple-stem oligonucleotide probes of the present disclosure are stem and loop structures formed by intramolecular hybridization of IHSs of each probe. Each IHS has a sequence complementary to another IHS of the probe, and complementary IHSs are of the same length. In the absence of target binding to the probe, complementary IHSs hybridize with each other forming a “stem” structure. The length of an IHS may be of any size that allows the sensor to work for accomplishing its stated purpose of detecting a target molecule, and may be determined by one of skill in the art provided with this disclosure without undue experimentation. In certain embodiments, internal hybridization sequence lengths will be in the range of about 5 to about 20 nucleotides, for example about 5, 6, 7, 8, 9, or 10 nucleotides per internal hybridization sequence.

The “loop” structures of each probe may be of any length suitable to the application, but may be between 3 to 20 nucleotides in length, for example, 4, 5, 6, 7, 8, 9, 10, 12, 14 or 16 nucleotides in length. The “triple-stem” conformation is the product of one or more of these stem and loop structures in one probe molecule. This is best explained through the aid of diagrams.

FIG. 1 depicts an exemplary embodiment of the present disclosure. The embodiment of FIG. 1 has six IHSs, 101, 103, 104, 105, 107, and 109. As presented, 101 hybridizes with 104 to form a first duplex, 105 hybridizes with 107 to form a second duplex, and 103 hybridizes with 109 to form a third duplex. This hybridization pattern results in the “triple-stem” conformation where the stem of the probe includes three portions of the single-stranded oligonucleotide sequence held together by the three self-complementary duplexes. In the depicted embodiment of FIG. 1, the first duplex is flanked by the second and third duplexes. In addition, the probe includes three loop structures. The first loop structure 102 is between IHS101 and IHS103, the second loop structure 106 is between IHS105 and IHS107, and the third loop structure 108 is between IHS107 and IHS109. The probe also includes a target binding sequence, which, as shown in the embodiment of FIG. 1, includes IHS103, IHS104, and IHS105.

In certain examples, the size of each stem structure may be different, as is also the case with loop structures. Limits on the size of each IHS pair, each loop, and the single-stranded linear probe length are not contemplated as being rigidly limited but are rather application-dependent. Optimal lengths for each of the probe components described herein may be determined without undue experimentation by one of skill in the art through the teachings of this specification. Lengths provided herein are exemplary only.

2. Fluorophore

Triple-stem oligonucleotide probes include a fluorophore attached to one end of the probe or at a central position in the probe sequence, so long as the position of the fluorophore allows the fluorophore to be positioned adjacent the quencher in the absence of target binding to the target binding sequence and away from the quencher when target binds to the target binding sequence. In some embodiments, as discussed in more detail below, the fluorophore may be attached to one end of the probe and the probe attached to the surface of a substrate at the other end of the probe. The fluorophore attached to the probe need not be a single molecule, but may include multiple molecules. Some exemplary embodiments of these alternatives are discussed in more detail below. The “end” of the triple-stem oligonucleotide probe possessing the fluorophore includes any nucleotide within one quarter of the total number of nucleotides in the probe from the terminal nucleotide. Alternatively, the end possessing the fluorophore includes the terminal 10, 9, 8, 7, 6, 5, 4, 3 or 2 nucleotides of the probe. Of course attachment may also be limited to the terminal nucleotide alone. The attachment of the fluorophore to the triple-stem oligonucleotide probe allows the fluorophore to be positioned in an alternate configuration at a distance away from the quencher in response to target specifically binding the probe, thereby generating a detectable signal.

The fluorophore may be synthetic or biological in nature, as known to those of skill in the art. More generally, any fluorophore can be used that is stable under assay conditions and that can be sufficiently suppressed when in close proximity to the quencher such that a significant change in the intensity of fluorescence of the fluorophore is detectable in response to target specifically binding the probe. Exemplary fluorophores include, but are not limited to, CAL Fluor Red 610 (FR610; Biosearch Technologies, Novato, Calif.), fluorescein isothiocyanate, fluorescein, rhodamine and rhodamine derivatives, coumarin and coumarin derivatives, cyanine and cyanine derivatives, Alexa Fluors (Molecular Probes, Eugene, Oreg.), DyLight Fluors (Thermo Fisher Scientific, Waltham, Mass.), and the like.

As explained above, the fluorophore may include multiple fluorophore molecules attached to a single probe. Advantages to such alternatives are known to one of skill in the art, and therefore only exemplary alternatives and advantages will be presented below for the advantage of the reader.

3. Quencher

Triple-stem oligonucleotide probes include a quencher attached at a central position away from the ends of the probe (i.e., at a position in the central portion of the probe sequence) or at one end of the probe, so long as the position of the fluorophore allows the fluorophore to be positioned adjacent the quencher in the absence of target binding to the target binding sequence and away from the quencher when target binds to the target binding sequence. The quencher attached to the probe need not be a single molecule, but may include multiple molecules. Some exemplary embodiments of these alternatives are discussed in more detail below. The attachment position of the quencher includes any nucleotide within the probe that positions the quencher in close proximity to the fluorophore in the absence of target specifically binding to the target binding sequence. The attachment of the quencher to the triple-stem oligonucleotide probe allows the quencher to be positioned in an alternate configuration at a distance away from the fluorophore in response to target specifically binding the probe, thereby allowing the fluorophore to emit a detectable signal.

As depicted for example in FIG. 1, the fluorophore 10 is attached to one end of the probe and the quencher is attached at a central position within the probe sequence. However, alternative embodiments are contemplated, for example, where the quencher is attached to one end of the probe and the fluorophore is attached at a central position within the probe sequence. Other alternative embodiments include, but are not limited to, probes where either the fluorophore or the quencher is attached to the 3′-end of the probe sequence, and probes where either the fluorophore or the quencher is attached to the 5′-end of the probe sequence.

The quencher may be synthetic or biological in nature, as known to those of skill in the art. More generally, any quencher can be used that is stable under assay conditions and that can sufficiently suppress the fluorescence of the fluorophore when in close proximity to the fluorophore such that a significant change in the intensity of fluorescence of the fluorophore is detectable in response to target specifically binding the probe. Exemplary quenchers include, but are not limited to Black Hole Quencher (BHQ; Biosearch Technologies, Novato, Calif.), Dabsyl (dimethylaminoazosulphonic acid), Qxl quenchers (AnaSpec Inc., San Jose, Calif.), Iowa black FQ, Iowa black RQ, and the like.

As explained above, the quencher may include multiple quencher molecules attached to a single probe. Advantages to such alternatives are known to one of skill in the art, and therefore only exemplary alternatives and advantages will be presented below for the advantage of the reader.

4. Multiplexing

In addition, in certain embodiments, multiplexing may be used. The terms “multiplex” or “multiplexing” as used herein refer to using multiple fluorescently distinct fluorophores, such that a single array may include multiple probes with different fluorophores. Fluorophores of these embodiments emit detectable signals at different wavelengths. Multiplexing facilitates the labeling of different probes (i.e., probes that comprise different target binding sequences) with fluorophores that emit different signals. In these embodiments, a mixture of differentially labeled probes may be contacted with a sample that comprises one or more different targets of interest. For example, a first probe that comprises a first target binding sequence and a first fluorophore may bind to a first perfectly matched target, as described above, and a second probe comprising a second target binding sequence and a second fluorophore may bind to a second perfectly matched target. Upon binding of the first perfectly matched target to the first target binding sequence of the first probe, a conformational change is induced such that a first signal of the first fluorophore is detectable. In addition, upon binding of the second perfectly matched target to the second target binding sequence of the second probe, a conformational change is induced such that a second signal of the second fluorophore is detectable. The first signal and the second signal may be detected, thus indicating the presence (or absence) of the first target and second target in the sample. In certain embodiments, multiplexing may be used in reactions comprising unbound triple-stem probes in solution, while in other embodiments, multiplexing may be used in systems comprising arrays or addressable arrays of triple-stem probes.

B. Triple-Stem Oligonucleotide Probe Targets

As explained above, probes of the present disclosure recognize nucleic acid targets through complementary base-pairing and are capable of use as a detector for targets that can be placed in solution. By way of example, targets that can specifically hybridize to the target binding sequence of the probe include perfectly matched targets. In these embodiments, the perfectly matched target hybridizes to the target binding sequence of the probe and induces a conformational change in the probe that positions the fluorophore at a distance away from the quencher, such that a signal of the fluorophore is detectable. In certain embodiments, targets that include one or more mismatched nucleotides, such as single-base mismatched targets, two-base mismatched targets, including three-base mismatched targets, or targets with more than three mismatched bases, do not specifically hybridize to the target binding sequence of the probe. In these cases, the mismatched targets will not specifically hybridize to the target binding sequence of the probe and the probe will remain in its triple-stem configuration such that the fluorophore is in close proximity to the quencher and the fluorescence of the fluorophore is suppressed by the quencher.

### Methods

### I. Detection of Targets Using Triple Stem Oligonucleotide Probe-Based Detectors

Provided are methods for detecting the presence of a target in a sample using the triple-stem oligonucleotide probe-based detectors. Aspects of the methods include bringing a sample suspected of containing a target into contact with a probe of the present disclosure under conditions that allow target that may be present in the sample to specifically bind to the target binding sequence of the probe. Binding of the target to the probe causes a conformational shift in the probe positioning the fluorophore at a distance away from the quencher sufficient to allow a signal of the fluorophore to be detectable. The signal detected by the detector may be optionally compared to control readouts for control samples that do not contain target or to results from samples that contain mismatched targets (i.e., negative controls). In other embodiments, the signal detected by the detector may be optionally compared to control readouts for control samples that contain target or a known amount of target (i.e., positive controls). Numerous alternative controls may be performed individually and in combination, as is known to those of skill in the art. For example, the control may be to challenge the probe with a surrogate solution absent the sample, and thus lacking target. Alternatively, the control may be a solution containing a “dummy” target that may have similarity to the actual target, but is normally not recognized and specifically bound by the probe under specific binding or “stringent” conditions.

In some cases, probes of the present disclosure may be contacted with a sample that contains perfectly matched target, while in other cases the probes of the present disclosure may be contacted with a sample that does not contain perfectly matched target. In these cases, the probes of the present disclosure are able to discriminate between samples that contain and that do not contain perfectly matched target. In certain embodiments, the difference between the detector reading in the presence of perfectly matched probe/target binding and in the absence of perfectly matched target (e.g. in the presence of mismatched target) may then be compared and a signal value determined for the target under the conditions employed. In some cases, a “discrimination factor” is calculated. The discrimination factor is the ratio of the net fluorescence intensity obtained in the presence of the perfectly matched target to that obtained in the presence of a mismatched target after subtraction of background fluorescence.

Suitable samples include bodily fluids, water, cell extracts, cell suspensions, secretions, solvents, and other aqueous and organic liquid solutions, suspension or emulsions capable of including the target of the probe of the detector. In certain embodiments, the probes of the present disclosure may be oligonucleotides that include a target binding sequence that specifically binds to target nucleic acids. In other embodiments, the probes may be aptamers that include a target binding sequence that bind a specific target molecule. In cases where the probes are aptamers, the targets may include, but are not limited to small molecules, proteins, cells, tissues, organisms, etc.

Particular methods of the disclosure are for detecting the presence of a target having a nucleotide sequence that is perfectly (i.e., 100%) complementary to a nucleic acid sequence of the target binding sequence of at least one probe species of the detector. The method involves contacting the triple-stem probe with a sample under hybridization conditions, whereby the target selectively hybridizes to the target binding sequence to form a target-probe hybrid. Target binding to the probe results in a detectable fluorescent signal as described previously. This signal is noted and optionally may be compared to the response of the detector to control samples or samples that include mismatched target as described above.

In other embodiments, particular methods of the disclosure are for detecting the presence of a single nucleotide polymorphism (SNP) in a target. In these embodiments, the target binding sequence of the probe includes a single nucleotide mismatch as compared to a wild-type sequence. The method includes contacting the triple-stem probe with a sample under hybridization conditions, whereby the target selectively hybridizes to the target binding sequence to form a target-probe hybrid. Target binding to the probe results in a detectable fluorescent signal as described previously. In these embodiments, since only perfectly matched targets will hybridize to the probe as described above, detecting the presence of the target-probe hybrid indicates the presence of a SNP in the target, where the SNP in the target is complementary to the single nucleotide mismatch in the target binding sequence.

For example, the methods of the present disclosure permit separate members of a gene family, related in sequence, to be discriminated in a complex sample of RNA or DNA, allowing the differential expression of such family members readily to be followed. The methods of the present disclosure similarly permit allelic variants of a single gene to be discriminated in a genomic sample, facilitating detection and scoring of single nucleotide polymorphisms (SNPs). The methods of the present disclosure improve discrimination in microarray-based analyses for measuring gene expression, analyzing genomic sequence variation, or sequencing by hybridization.

A. Reaction Conditions and Detection Methods

The methods disclosed herein may be carried out in any reaction medium that allows specific binding between probe and, if present, target as defined herein. In cases where the sample contains perfectly matched target, specific binding between the target binding sequence of the probe and the perfectly matched target is favored over intramolecular hybridization between the internal hybridization sequences of the probe. In cases where the sample contains mismatched target, intramolecular hybridization between the internal hybridization sequences of the probe is favored over binding between the probe and mismatched target. In some cases, the reaction medium includes ionic species that increase the ionic strength of the reaction medium. In aqueous reaction media the ionic species may be simple salts but may include more complex species, depending on the reaction media employed, as will be readily appreciated by one of skill in the art. Although some of the binding reactions discussed herein may be performed without regard to ionic strength, reaction media may have an ionic strength between about 0.001N and about 5N or between about 0.01N and 0.5N, with common ionic strengths including, but not limited to, 0.03N, 0.04N, 0.05N, 0.06N, 0.07N, 0.08N, 0.09N, 0.1N, 0.15N, 0.2N, 0.25N, 0.75N, 0.9N and 1N. Salts of magnesium, potassium, calcium, and/or manganese ions may be paired with halogen counter ions. In addition, phosphate, sulphate, carbonate, and the like may be used. The list of suitable ionic species for use in the present disclosure is lengthy, but suitable ionic species will be readily evident to one of skill in the art.

Binding reactions involving the probes disclosed herein may be carried out in the presence of agents and additives that promote the desired specific binding, diminish nonspecific background interactions, inhibit the growth of microorganisms, or increase the stability of the probe and/or target. For example, one may add up to 10% by weight or volume (based on the amount of aqueous environment), such as from about 1% or 2% to about 10% of one or more polyols. Representative polyols include glycerol, ethylene glycol, propylene glycol, sugars such as sucrose or glucose, and the like. One may also add similar levels of water soluble or water dispersible polymers such as polyethylene glycol (PEG), polyvinyl alcohol, or the like. Another representative additive is up to about 1% or 2% by weight (again based on the liquid substrate) of one or more surfactants such as triton X-100 or sodium dodecyl sulfate (SDS). Numerous specific binding conditions have been described and are well known in the art. Many such conditions are useful for practicing the methods and systems of the present disclosure.

Binding reactions of the disclosure may be carried out at ambient temperature, although any temperature in the range allowing specific binding may be used. For instance in some embodiments, the temperature range is from about 5° C. to about 45° C. In some cases, the salt concentration of the binding reaction medium approximates physiological salt concentration. For example, the salt concentration may be between 10 mM to 300 mM, such as 50 mM to 250 mM, including 100 mM to 200 mM. In particular cases, the salt concentration may be about 150 mM. In addition, in particular embodiments, the pH of the binding reaction medium is about physiological pH. For example, the pH may be between 4 to 10, such as 5 to 9, including 6 to 8. In particular cases, the pH may be about 7. In certain embodiments, the reaction medium has a salt concentration of about 150 mM and a pH of about 7. For convenience, conditions are typically chosen to allow specific binding to occur as rapidly as possible. Binding times as short as minutes (e.g. about 1 to 30 minutes) may be employed. By way of example, times of up to 45, 60, 90, 120, 150, or 180 minutes, or longer may be used. Typical binding times are from about 5 to about 45 minutes. Temperatures and times of target/probe incubation to achieve satisfactory results may be determined empirically, e.g. CoT analysis or other methods of predicting binding conditions as are known to those of skill in the art. For instance, reaction conditions may be employed that allow for preferential binding of the target to the target binding sequence of the probe rather than intramolecular hybridization between the internal hybridization sequences of the probe.

B. Microelectrodes and Arrays

The probes and systems of the present disclosure are well suited for applications in electronic gene detection arrays. To this end, biomaterials may be deposited onto a substrate surface in the form of an array or an addressable array. In one embodiment, the microelectrodes are arrayed in the format of N features, with each feature forming a detector having a unique triple-stem probe of the disclosure. Each detector is independently addressable, thereby enabling detection of N different perfectly matched targets.

An “array”, as the term is used herein, includes any one-dimensional, two-dimensional or substantially two-dimensional (as well as a three-dimensional) arrangement of addressable regions bearing a particular chemical moiety or moieties (such as ligands, e.g. biopolymers such as polynucleotide or oligonucleotide sequences (nucleic acids) associated with that region. Arrays may be referred to as addressable. An array is “addressable” when it has multiple regions of different moieties (e.g. different polynucleotide sequences) such that a region (i.e., a “feature” or “spot” of the array) at a particular predetermined location (i.e., an “address”) on the array will detect a particular target or class of targets (although a feature may incidentally detect non-targets of that feature). These regions may or may not be separated by intervening spaces which do not carry any polynucleotide (or other biopolymer or chemical moiety of a type of which the features are composed). The nucleic acids may be covalently attached to the arrays at any point along the nucleic acid chain, but are generally attached at one of their termini (e.g. the 3′ or 5′ terminus).

Any given substrate may carry one, two, four or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features.

Thus, in certain embodiments, the triple-stem probes disclosed herein may be immobilized on a substrate, such that the probes form an addressable array of probes. The triple-stem probes that comprise the addressable array may be identical, or in other embodiments, a plurality of different triple-stem probes (i.e., probes with different target binding sequences) may comprise the addressable array. In these embodiments, the composition and location of each probe is known, such that the sequence of any targets binding to the array is readily obtained because the sequence of the probes at each location in the addressable array is known (i.e., the sequence of the target bound to the array is complementary to the target binding sequence of the probe the target is bound to).

With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 590 nm or 610 nm. The substrate may be porous or non-porous. The substrate may have a planar or non-planar surface.

The term “substrate” as used herein refers to a surface upon which probes, e.g. an array, may be immobilized. Glass slides may be used as the substrate, although fused silica, silicon, plastic and other materials are also suitable.

As demonstrated in the following examples, the oligonucleotide triple-stem probe-based detectors claimed herein are both sensitive and highly selective. The detectors employing the triple-stem probes may be constructed using synthesis techniques well known to those of skill in the art, such as but not limited to, drop deposition using ink-jets, or the like, or light directed synthesis fabrication.

### Kits

Also provided are kits that find use in practicing the subject methods, as described above. For example, kits and systems for practicing the subject methods may include one or more systems of the present disclosure, which may include one or more triple-stem probes. As such, in certain embodiments the kits may include a solution or suspension of the probes in an aqueous or other compatible solution. In other embodiments, the kits may include one or more probes immobilized on the surface of a substrate forming an addressable array of probes.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another means would be a computer readable medium, e.g., diskette, CD, DVD, computer-readable memory, etc., on which the information has been recorded or stored. Yet another means that may be present is a website address which may be used via the Internet to access the information at a removed site. Any convenient means may be present in the kits.

As can be appreciated from the disclosure provided above, the present disclosure has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLES

### Materials and Methods

All chemicals were purchased from Sigma-Aldrich, Inc. (Saint Louis, Mo.) and used without further purification. The fluorophore/quencher-labeled DNA oligonucleotide probes were synthesized by Biosearch Technologies, Inc. (Novato, Calif.) and purified by C18 HPLC, and confirmed by mass spectrometry. The triple-stem SNP sensor was composed of a single DNA strand (1) that was modified with a CAL Fluor Red 610 (FR610) fluorophore at the 3′ terminus and a Black Hole Quencher (BHQ) at an internal position. The molecular beacon (MB) structure (5) was a single 7-bp stem, in which the binding sequence was present in the entire loop as well as part of the stem. The pseudoknot structure (6) had two 7-bp stems in which the first stem's loop formed one strand of the second stem, and the binding sequence was only located in the 5′ loop.

The sequences of these modified oligomers were as follows:

(1) 5′-AGGCTGGATTTTTTATTTACCTTTTTTTAGGTAAAA-(BHQ)-
CGACGGCCAGCCTTTTTTTTTTTTTCCGTCGT-(Cal Fluor 610)-
3′
(5) 5′-(Cal Fluor 610)-
AGGCTGGAGGTAAAACGACGGCCAGCCT-(BHQ)-3′
(6) 5′-GGCGAGGTAAAA-(BHQ)-
CGACGGCCAGCCTCGCCGTTTTTTTTTTTTTTTTTGCCGTCG-T-
(Cal Fluor 610)-3′

The perfectly-matched targets (i.e., PM targets) and mismatched DNA targets (i.e., 1 MM and 2 MM targets) were purchased from Integrated DNA Technologies Inc. (Coralville, Iowa), and were purified by HPLC. The sequences of these DNA targets were as follows (mismatched nucleotides are indicated in bold):

| | |
|---|---|
| 15-mer DNA targets: | |
| PM target: | 5′-CTGGCCGTCGTTTTA-3′; |
| 1 MM target: | 5′-CTGGCCGTAGTTTTA-3′ |
| 17-mer DNA targets: | |
| PM target (2): | 5′-GCTGGCCGTCGTTTTAC-3′ |
| 1 MM target (3): | 5′-GCTGGCCCTCGTTTTAC-3′ |
| 2 MM target (4): | 5′-GCTGGCCCCCGTTTTAC-3′ |
| 19-mer DNA targets: | |
| PM target: | 5′-GGCTGGCCGTCGTTTTACC-3′ |
| 1 MM target: | 5′-GGCTGGCCCTCGTTTTACC-3′ |

### Determination of Melting Temperatures (Tₘ)

Fluorescence melting curves of different modified probes were measured at 610 nm with a Varian (Palo Alto, Calif.) Cary 100 spectrometer equipped with a Peltier block. A degassed aqueous solution containing 1 mM phosphate buffer (PB), 30 mM MgCl₂ and 1 mM NaCl (pH 7) was used as the hybridization solution. The oligonucleotides were mixed at a 1:1 ratio (v/v) in the degassed hybridization buffer at room temperature, and the solutions were adjusted to a final volume of 100 μl. The duplexes were formed between the modified beacon (5), pseudoknot (6) or three-stemmed probe (1) and 17-mer targets. Prior to analysis, the samples were heated to the maximum temperature of 95° C. for 10 minutes and cooled to starting temperature of 20° C. Melting curves were recorded at a rate of 0.5° C./min. The same hybridization buffer was used for all experiments. Melting temperatures (Tₘ) were observed to be 32.0° C., 45.4° C. and 80.2° C. for the MB, pseudoknot and triple-stem probes, respectively.

### Measurements of Discrimination Factors

Solution mixtures containing the modified beacon (5), pseudoknot (6) or three-stemmed probe (1) (0.5 μM) and variable concentrations of PM or 1 MM targets in 100 μl of hybridization buffer were incubated at room temperature for 3 hours and then subjected to fluorescence emission spectrum measurements. Experiments were performed at an excitation wavelength of 590 nm and emission scan of 595-800 nm. Fluorescence intensities at 610 nm were used for calculation of discrimination factors.

The SNP detection performance of a triple-stem probe was tested against two alternative FR610 fluorophore/BHQ quencher labeled probes, each of which contained different secondary structure attributes (see Table 1, scheme). The first was a molecular beacon (MB) structure (5) with a single 7-bp stem, in which the binding sequence was present in the entire loop as well as part of the stem. The second was a pseudoknot structure (6) with two 7-bp stems in which the first stem's loop formed one strand of the second stem, and the binding sequence was only located in the 5′ loop. The specificity of the triple-stem probe was significantly greater than that of the MB and pseudoknot probes as seen by comparison of the discrimination factors. The single-mismatch discrimination factor is the ratio of the net fluorescence intensity obtained with the perfectly-matched target to that obtained with the single-base mismatched target after subtraction of background fluorescence. By this metric, a larger discrimination factor is indicative of greater specificity. When challenged with a 17-base target, both the MB (5) and pseudoknot (6) probes exhibited relatively poor specificity, with discrimination factors of 1.5 and 2.9 respectively (see Table 1). In contrast, a discrimination factor of 28.4 was observed for the triple-stem probe (1) (see Table 1) under the same conditions. Moreover, the triple-stem probe also had a higher discrimination factor for shorter (e.g. 15-base) or longer (e.g. 19-base) targets which did not trigger significant above-background fluorescence in the MB and pseudoknot probes (see Table 1). Thus, in certain embodiments, the specificity of the triple-stem probe response may facilitate the detection of single-nucleotide substitutions in targets of different lengths.

Table 1, shown below, presents discrimination factors of various probe types against single-base mismatches located in the middle position of 15-, 17-, and 19-base DNA targets. The single-mismatch discrimination factor is defined as the ratio of the net fluorescence intensity obtained with the perfectly-matched target to that obtained with the single-base mismatched target after subtraction of background fluorescence.

The ability of the triple-stem probe to discriminate against a wide variety of single-base mismatches located at different positions within the sequence of a 17-base DNA target was also tested. Discrimination factors ranging from 5.6 to 28.4 (see Table 2) were observed. The highest level of discrimination was obtained with duplexes containing a C/C mismatch; conversely, the lowest discrimination level was observed with the A/A mismatched duplex. While strong discrimination was reproducibly observed for all single-base mismatches, the discrimination factor may depend on the identity of the mismatched base-pair as well as the identity of its nearest neighbors.

Table 2, shown below, presents discrimination factors of the triple-stem probe for single-base mismatched targets differing from the 17-base perfect match target (2) (5′-GCTGGCCGTCGTTTTAC-3′) by a single nucleotide at various sites (mismatches marked in bold).

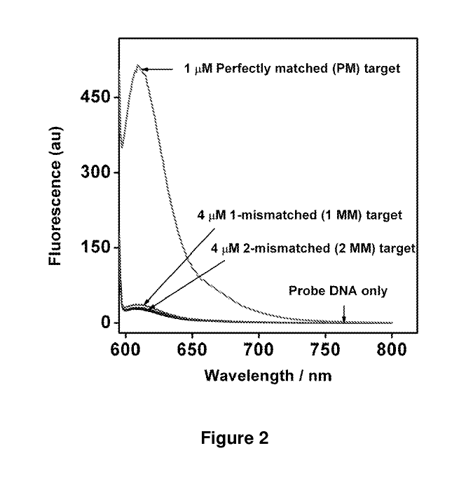

The triple-stem SNP sensor was composed of a single DNA strand (1) that was modified with a CAL Fluor Red 610 (FR610) fluorophore at the 3′ terminus and a Black Hole Quencher (BHQ) at an internal position. At room temperature, the modified, 68-base probe self-hybridized into three separate, seven base-pair (bp) Watson-Crick stems that formed a discontinuous, 21-base double helix (see FIG. 1, left). In the absence of target, this relatively rigid triple-stem structure held the fluorophore in close proximity to the quencher, resulting in very limited fluorescence (see FIG. 2, probe only). Upon hybridizing to a perfectly-matched target (PM; 2) the triple-stem structure was disrupted, which separated the fluorophore/quencher pair (see FIG. 1, upper right) and induced a 29-fold increase in emission intensity (see FIG. 2, PM target). In contrast, when the sensor was challenged with target containing a single-base mismatch located in the middle of the sequence (1 MM; 3), only a 1.3-fold increase in emission intensity was observed, even at a four-fold higher concentration of mismatched target (see FIG. 2, 1 MM target); a two-base mismatched target (2 MM; 4) did not produce any detectable increase in fluorescence (see FIG. 2, 2 MM target).

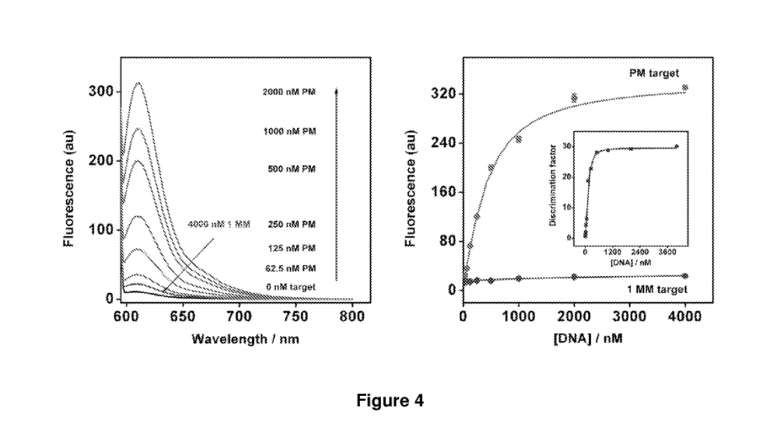

In order to characterize the presently disclosed triple-stem probe's discrimination capacity between low concentrations of perfectly-matched target versus a higher concentration of single-base mismatched target, titration experiments of the perfectly-matched and single-base mismatched targets into solution containing the triple-stem probe at room temperature were performed. The fluorescence of the DNA probe itself was minimal (see FIG. 4, left, 0 nM target), but fluorescence intensity significantly increased in a concentration-dependent manner in the presence of perfectly-matched target, with the discrimination factor peaking at about 30 (see FIG. 4, right, inset). In contrast, a 1.5-fold increase in fluorescence intensity was observed in the presence of 4 μM single-base mismatched target see (FIG. 4 left, 1 MM). Thus, the triple-stem probe was sensitive enough to achieve robust single-nucleotide discrimination over a wide target concentration range (see FIG. 4, right), up to 300 μM (data not shown); for example, the triple-stem probes showed a discrimination factor of 4 in a comparative analysis with 32 nM of each target, and a discrimination factor of 5 for an analysis of 125 nM of perfectly-matched target versus 4 μM single-base mismatched target (see FIG. 4, right, inset).

FIG. 4, left, shows emission spectra of the triple-stem probe (1) (0.5 μM) only, probe-single-base mismatched target (3) duplexes, or probe-perfectly-matched target (2) duplexes at different concentrations, recorded at room temperature. FIG. 4, right, shows a calibration curve of perfectly-matched target (2) and single-base mismatched target (3) for the triple-stem probe (1). The signal change demonstrates sensitive discrimination ability over wider target concentration range. The inset shows the dependence of discrimination factor of 17-base targets in the presence of 0.5 μM of the triple-stem probe.

### Fluorescence Denaturation Experiments

Thermal melting curves were obtained with a Varian Cary Eclipse spectrometer equipped with a Peltier block, using quartz fluorescence cuvettes (4×10 mm; Sub-micro, 50 μl), and with the following settings: λₑₓ=590 nm, λₑₘ=610 nm, 5 nm slit for excitation and emission, PMT detector voltage=650V. The hybridization of the modified three-stemmed probe (1) (0.5 μM) with 1.0 μM 17-mer PM target, or 4 μM 1 MM or 2 MM targets in 100 μl of the degassed hybridization buffer was performed at room temperature for 3 hours. Melting curves were recorded at a rate of 0.5° C./min at an average interval time of 0.1 s, starting at 20° C. and finishing at 95° C. Before denaturation experiments, a calibration of cuvettes was done in order to obtain the same fluorescence intensity of probe for all samples.

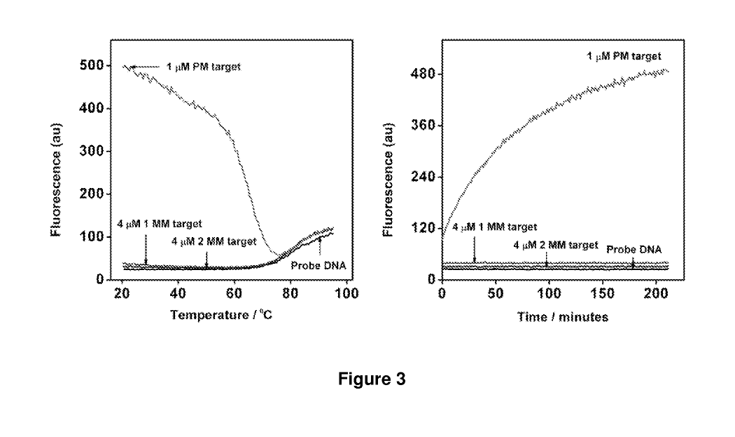

The triple-stem probe sensors function over a wide temperature range and exhibit SNP discrimination from room temperature up to about 60° C., or more. Denaturation experiments were performed by monitoring the fluorescence change as a function of temperature in the absence of targets and in the presence of perfectly-matched, single-base mismatched or two-base-mismatched targets (see FIG. 3, left). At low temperatures, the probe hybridized with the perfectly-matched targets, giving rise to significantly increased fluorescence. At higher temperatures, the duplex structure was destabilized and the released probe was able to re-fold into the native triple-stem structure, resulting in significantly diminished fluorescence intensity. For perfectly-matched targets, this transition from the target-probe duplex to the self-complementary triple-stem structure occurred at approximately 65° C. As the temperature was raised further (e.g. above 82° C.), the folded probes melted into random coils in which quenching efficiency was reduced, resulting in a small increase in fluorescence at the highest temperatures (see FIG. 3, left, PM target). In contrast, little fluorescence was observed in the presence of the single-base or two-base-mismatched targets at low temperature (see FIG. 3, left, 1 MM or 2 MM targets), while the high temperature dependencies were similar to the perfectly-matched target. The same transition temperatures were observed in the presence of mismatched targets or in the absence of targets (see FIG. 3, left, probe only).

FIG. 2 shows emission spectra of the triple-stem probe (1) (0.5 μM) following incubation at room temperature with a perfectly-matched (PM) target (2), single-base mismatched (1 MM) target (3), two-base mismatched (2 MM) target (4), or in the absence of target. Fluorescence intensity was enhanced 29-fold in the presence of the perfectly-matched target. In contrast, mismatched targets gave almost no fluorescence increase. The fluorescence signal was obtained at λₑₓ=590 nm and λₑₘ=610 nm, and all targets were 17 bases in length.

FIG. 3, left, shows thermal denaturation curves of the triple-stem probe (1) (0.5 μM) only, or hybridized with a perfectly-matched (PM) target (2), a single-base mismatched (1 MM) target (3), or a two-base-mismatched (2 MM) target. The discrimination ability of the triple-stem SNP sensor was maintained up to 60° C.

### Kinetic Experiments

Kinetic experiments were measured at room temperature using a Varian Cary Eclipse spectrometer, with the same experimental conditions used in the fluorescence denaturation experiments described above. All measurements were carried out with 0.5 μM probe in the presence of 1.0 μM 17-mer PM target, or 4 μM 17-mer 1 MM or 2 MM targets (100 μl as a total reaction volume). Solution containing only the labeled probe was used as one of the control experiments. Fluorescence intensities were immediately recorded after adding target molecules.

To monitor the response time of the triple-stem probe, real-time kinetic measurements at room temperature were performed, monitoring the fluorescence intensity before and after the addition of 1 μM perfectly-matched or 4 μM single- or two-base-mismatched targets. At room temperature, the probe stably maintained its self-complementary, fluorescence-quenching structure (see FIG. 3 right, probe only). Upon addition of the perfectly-matched target, a significant increase in fluorescence over time was observed (see FIG. 3, right, PM target), while almost no signal increase was detected in the presence of mismatched targets (see FIG. 3, right, 1 MM or 2 MM targets). In comparison to other commonly-employed SNP detection strategies, such as enzyme-mediated methods, the triple-stem probe produced a relatively rapid response: a discrimination factor of 16 was obtained after a 30-minute hybridization, with signal saturation occurring at a discrimination factor of 28.4 after about 3 hours (see FIG. 3, right).

FIG. 3, right, shows a graph of the kinetics of the triple-stem probe (1) (0.5 μM) only, or hybridized with perfectly-matched (PM), single-base (1 MM) or two-base-mismatched (2 MM) targets, monitored at room temperature. A discrimination factor of 16 was obtained after a 30-minute reaction.

The preceding merely illustrates the principles of the disclosure. All statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

That which is claimed is:

1. A system for detecting a target in a sample comprising:
a single-stranded oligonucleotide probe comprising:
(i) a target binding sequence;
(ii) a first hybridization sequence;
(iii) a second hybridization sequence;
(iv) a third hybridization sequence;
(v) a fourth hybridization sequence;
(vi) a fluorophore; and
(vii) a quencher,
wherein
in the absence of target binding to the target binding sequence, the first hybridization sequence and the second hybridization sequence form a first duplex and the third hybridization sequence and the fourth hybridization sequence form a second duplex such that the probe is in a triple-stem configuration and the fluorophore is positioned adjacent the quencher, and
in the presence of target binding to the target binding sequence, formation of duplexes between the hybridization sequences is inhibited by specific interaction of the target with the target binding sequence such that the probe is configured to position the fluorophore away from the quencher such that a signal of the fluorophore is detectable.

2. The system of claim 1, wherein the target binding sequence comprises at least a portion of the first hybridization sequence.

3. The system of claim 1, wherein the target binding sequence comprises at least a portion of the second hybridization sequence.

4. The system of claim 1, wherein the target binding sequence comprises at least a portion of the second hybridization sequence and at least a portion of the third hybridization sequence.

5. The system of claim 1, wherein the first duplex and the second duplex are adjacent each other when the probe is in the triple-stem configuration.

6. The system of claim 1, further comprising a fifth hybridization sequence and a sixth hybridization sequence.

7. The system of claim 6, wherein in the absence of target binding to the target binding sequence, the fifth hybridization sequence and the sixth hybridization sequence form a third duplex.

8. The system of claim 7, wherein the first duplex is flanked by the second duplex and the third duplex.

9. The system of claim 7, wherein the second duplex and the third duplex are separated by a hairpin structure.

10. The system of claim 7, wherein the first duplex, the second duplex, and the third duplex together comprise about 10 to about 30 base pairs.

11. The system of claim 10, wherein the first duplex, the second duplex, and the third duplex together comprise about 21 base pairs.

12. The system of claim 11, wherein the first duplex, the second duplex, and the third duplex together comprises 21 base pairs.

13. The system of claim 6, wherein the target binding sequence comprises at least a portion of the second hybridization sequence, at least a portion of the third hybridization sequence, and at least a portion of the sixth hybridization sequence.

14. The system of claim 1, wherein the target binding sequence only hybridizes to a nucleic acid target when perfectly complementary to the target.

15. The system of claim 1, wherein the target binding sequence has a discrimination factor of about 5 or more.

16. The system of claim 1, wherein the target binding sequence comprises about 10 to about 30 contiguous nucleotides complementary to the target.

17. The system of claim 16, wherein the target binding sequence comprises about 15 to about 19 contiguous nucleotides complementary to the target.

18. The system of claim 16, wherein the target binding sequence comprises 17 contiguous nucleotides complementary to the target.

19. The system of claim 1, wherein the quencher is attached to the probe at a position within the target nucleotide sequence, and wherein the fluorophore is attached to the probe at an end of the probe sequence.

20. The system of claim 1, wherein the fluorophore is attached to the probe at a position within the target nucleotide sequence, and wherein the quencher is attached to the probe at an end of the probe sequence.

21. The system of claim 1, wherein the probe is immobilized on a surface of a substrate.

22. The system of claim 21, wherein the substrate comprises an addressable array of a plurality of the probes.

23. A method for detecting a target in a sample comprising:
(a) contacting a single-stranded triple-stem probe of claim 1 with the sample under hybridization conditions, whereby the target selectively hybridizes to the target binding sequence to form a target-probe hybrid; and
(b) detecting the presence or absence of the target-probe hybrid, wherein the detecting comprises detecting fluorescent emission from the fluorophore.

24. The method of claim 20, wherein the concentration range of target in the sample is from about 1 nM to about 300 nM.

25. The method of claim 22, wherein the concentration range of target in the sample is from about 2 nM and about 150 nM.

26. A method for detecting the presence of a single nucleotide polymorphism in a target, comprising:
(a) contacting a single-stranded triple-stem probe of claim 1 with a sample comprising the target under hybridization conditions, wherein the target binding sequence comprises a single nucleotide mismatch, and whereby the target selectively hybridizes to the target binding sequence to form a target-probe hybrid; and
(b) detecting the presence or absence of the target-probe hybrid, wherein the presence of the target-probe hybrid indicates the presence of a single nucleotide polymorphism in the target, wherein the single nucleotide polymorphism in the target is complementary to the single nucleotide mismatch in the target binding sequence.
